(19) United States
(12) Patent Application Publication
(10) Pub. No.: US 2019/0133839 A1
(43) Pub. Date: May 9, 2019

(54) Protective Shield Blocker

(71) Applicant: Alesha Puckett, Lithonia, GA (US)

(72) Inventor: Alesha Puckett, Lithonia, GA (US)

(21) Appl. No.: 15/803,003

(22) Filed: Nov. 3, 2017

(51) Int. Cl. A61F 13/475; A61F 13/472; A61F 13/15

(52) U.S. Cl. CPC A61F 13/4758; A61F 13/8405; A61F 13/15203; A61F 13/47236

(57) ABSTRACT

The present invention is a disposable feminine hygiene product specially designed to contain menstrual flow leakage in the event a person is encountering heavy or irregular menstrual flow. The Protective Shield Blocker ensures that irregular menstrual flow will be concealed. In an alternative embodiment, the present invention can also be sold in a separate configuration that is ideal for other brands of sanitary products.

### FIELD OF INVENTION

The subject disclosure relates to an article for absorbing body exudate. More particularly, the present disclosure relates to a feminine hygiene product for preventing leakage due to irregular menstrual flow by using a specially designed embodiment.

### COPYRIGHT AND TRADEMARK NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever. Trademarks are the property of their respective owners.

### BACKGROUND

The uterus is normally in a straight vertical position. A tipped uterus is when the uterus is tilted backwards toward the back of the pelvis. Women with a retroverted uterus during the menstrual cycle tend to bleed both frontwards and backwards. This condition affects more than 30% of women worldwide. The cause in most cases are genetic and is entirely normal. Other factors that can contribute to this condition is Pregnancy, Menopause, and Reproductive Problems. Women who have this condition tend to bleed in an irregular direction which then causes soiling of clothing. With the present invention, it will be a process of elimination because the invention will act as a catalyst between a sanitary product and the undergarment. Traditionally, women have relied on products such as disposable sanitary napkins to help control menstrual flow. Disposable sanitary napkins have been inconvenient and unhygienic for individuals who encounter heavy menstrual flow or females who were born with an retroverted or tilted uterus that causes their menstrual cycle to flow in an abnormal direction that leaves conventional sanitary napkins ineffective. This ordeal can result in a person feeling uncomfortable while performing such activities as exercising, working, or even sleeping as an example.

Users who live with this condition whether caused by genetics or other unrelated health concerns are forced to deal with the inconvenience of conventional sanitary napkins. Often, users who have heavy menstrual cycles or irregular menstrual flow have to place tissue or similar material adjacent to the sanitary napkin to prevent leakage which can be time consuming and embarrassing. Although the use of such alternative means is necessary, it can be inconvenient for individuals who use conventional feminine sanitary napkins.

Prior art solutions to similar problems have been focused on using attachment straps such as belts that attach to the user's skin in order to collect fluids. These solutions were not ideal for the user due to the un-comfort caused by form and fit of the pad surface to a user's body as they move.

### BRIEF SUMMARY

The proposed invention is a disposable feminine hygiene product that achieves a dynamic body fit which allows users to benefit from a broader range of protection to combat leakage due to menstrual cycles. More specifically, in accord with one aspect of the Protective Shield Blocker, the proposed hygiene product includes an absorbent pad surface to block menstrual back flow by conveniently placing the Protective Shield Blocker between the user's buttocks for maximum coverage which is where menstrual backflow usually occurs. When the user experiences irregular or heavy menstrual flow, the conventional means of preventing back flow by using sanitary napkins is not affective at protecting against irregular leakage which often occur along the rear portion of sanitary napkins due to limited coverage. The Protective Shield Blocker is positioned specifically in the perineal area to quickly absorb back flowing leakage; this will protect undergarments and outer clothing in the event of such irregular menstrual flow. The key to this novel invention is that it covers areas of a person's body that have not been previously addressed due to a retroverted uterus. More specifically, the current embodiment provides a pad surface and a cord made of flexible material that protects undergarments and outer clothing. Where menstrual flow has leaked, the Protective Shield Blocker will form a barrier by utilizing the absorbent pad surface that acts as an additional barrier to existing sanitary products. The Protective Shield Blocker will change the way that feminine products are currently used among young girls and women who require special attention as a result of irregular menstrual flow or heavy bleeding.

### BRIEF DESCRIPTION OF THE DRAWINGS

Certain illustrative embodiments and advantages may be best understood by reference detailed description that follows taken in conjunction with the accompanying drawings in which:

FIG. 1 is a diagram of a side-view consistent with certain embodiments of the present invention. The schematic illustrates a fully assembled protective shield blocker, that demonstrates how the pad surface connects to the cord.

FIG. 2 is a diagram of a front-view of the proposed invention consistent with certain embodiments of the present invention.

FIG. 3 is a diagram of a cross-sectional view consistent with certain embodiments of the pad surface of the protective shield blocker.

FIG. 4 is a diagram of a bottom view consistent with certain embodiments of the pad surface of the protective shield blocker

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one, or more than one. The term “plurality”, as used herein, is defined as two, or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an exemplary embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Reference throughout this document to “space” or similar terms means a particular area that make up a structure and is described in connection with the position or the embodiment that is included in an area between two or more components of the present invention. Thus, the appearances of such phrases or in various places throughout this specification are not necessarily all referring to the same embodiment. Furthermore, “space” may be described in any suitable manner by one or more embodiments without limitation.

Reference throughout this document to “arresting” or similar terms indicate a means of stopping leakage from escaping a certain section of the present invention. Thus, the appearances of such phrase or in various places throughout this specification are not necessarily all referring to the same embodiment. Furthermore, “arresting” may be described in any suitable manner by one or more embodiments without limitation.

Reference throughout this document to Protective Shield Blocker or similar terms refer to a feminine hygiene product which has a long and circular body that serves as a pad surface for absorbing excess menstrual flow and a cord extending vertically from the base of the pad surface. Thus, the appearance of similar phrases throughout this specification may not be limited to just the term Protective Shield Blocker but when the description refers to a means of absorbing menstrual flow then it may be inferred that the description refers to the Protective Shield Blocker.

Reference throughout this document to “panel” refers to the portions of the Protective Shield Blocker that make up the inner and outer wall of the product. Furthermore, the particular features, structures, or characteristics may be combined in any suitable manner in one or more embodiments without limitations.

Reference throughout this document to attachment surface refers to any method of securing the feminine hygiene product to an additional brand of sanitary products such as a sanitary napkin but is not limited to this example. Therefore, where ever this specification refers to any means of attachment it will not be limited to any specific means of attachment but rather any number of attachment methods may be combined to create an attachment for purposes of the present embodiment.

Reference throughout this document to “cord” refers to an attachment for connecting the pad of the present invention to any number of existing products intended for absorbing human exudate. Therefore, wherever this specification refers to a cord it will not be limited to any specific means of attachment, material or structure but rather any number of attachment methods, materials or structures may be combined to create an attachment for purposes of the present embodiment. In a non-limiting example, the absorbent pad can be sold separately and attached to other sanitary products for both medical as well as non-medical purposes.

Reference throughout this document to “existing sanitary products” refers to any number of traditional sanitary products that are commonly used during menstrual cycles. Therefore, wherever this specification refers to existing sanitary products it will not be limited to any specific product but rather any number of sanitary products and structures may be combined for purposes of the present embodiment. In a non-limiting example, the existing sanitary product can be in reference to a sanitary napkin or tampon but is not limited to these examples.

Reference throughout this document to “landing” refers to a structure that is used to attach the cord to an additional sanitary product. Therefore, wherever this specification refers to a landing it will not be limited to any specific platform, size or shape but rather any number of platforms, sizes and shapes may be combined for purposes of the present embodiment. In a non-limiting example, the landing can be a flat rectangular platform but is not limited to this example.

The material that a feminine product is made of is an essential aspect of the appealability of the product because it can be a predictability of a user's experience when wearing the product while sleeping, exercising, or just sitting. From a convenience perspective, the aspects of the product's comfort which affects the user's sensory factors are an important attribute for effectively presenting a more inviting experience during an otherwise uncomfortable time of the month. In a non-limiting example, because odors emit from surfaces of such products then a combination of scents and special liners may be used to deal with odors and release scents that are pleasant to the user. Furthermore, in an additional non-limiting example, the liner of the absorbent pad surface can be made to release pleasant hypoallergenic fragrances to help eliminate odors.

In the exemplary embodiment, the current invention may consist of a method of attaching the cord structure to other surfaces for an alternate use. The method may encompass any number of commonly used methods in which similar products have been attached. For the sake of the current invention the method of attachment will not be limited to a certain method.

In an additional embodiment, the Protective Shield Blocker can be marketed as a different configuration in that the absorbent pad portion can be separately sold in various shapes and sizes to consumers such as maxi, overnight, and mini. The Protective Shield Blocker uses a soft dry weave material that fully fits along the curves of a user's perineal area by contouring to a user's skin to create a seal which prevents menstrual flow from escaping as a result of back flow. The absorbent pad surface allows the user to move freely without feeling uncomfortable. In an alternate embodiment, the Protective Shield Blocker may also be utilized for a variety of medical purposes for both humans as well as veterinarian purposes. As a non-limiting example, a version of the Protective Shield Blocker can be used on male patients during post-surgery for such ailments as prostate cancer or other medical purposes such as absorbing human exudate from male's penis or generally in the male genitalia area.

In a non-limiting example, the pad surface may include absorbent core material, and a scented hypoallergenic component that can be evenly distributed to act as a releasing agent for a more pleasant scent. The pad component may also include a water proof laminated outer surface that in a non-limiting example may be made out of non-woven material which incorporates an appealing design. This outer surface helps to prevent any fluids from leaving the pad mainly through the surface of the external surface which could be a significant inconvenience for the user. This is important to allow a main barrier of protection in an effort to contain menstrual flow inside the absorbent core of the Protective Shield Blocker as it exits the body. The optional cord is an important aspect of the configuration because it acts as means of attaching the pad component to existing sanitary products for increased options for consumers.

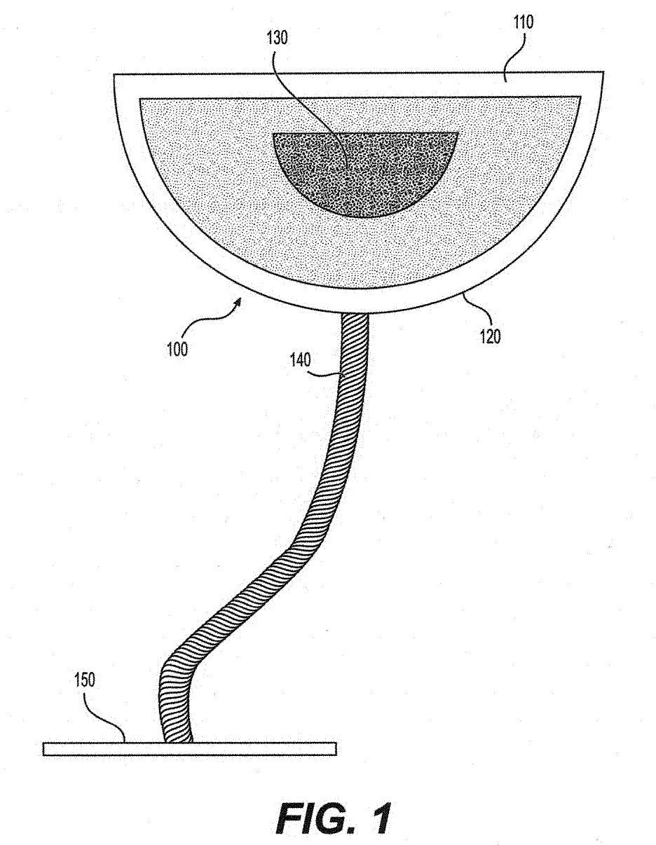

Turning now to FIG. 1, this figure presents a side-view of the Protective Shield Blocker 100 for an exemplary illustration to include the flexible cord 140. The Protective Shield Blocker 100 can be securely attached to other existing sanitary products by attaching the cord 140 by use of the landing 150. The Protective Shield Blocker has an upper permeable section 120 and a lower impermeable section 110. The schematic further presents an absorbent material 130 to ensure that menstrual flow is adequately contained. The wearer must attach the landing 130 to an existing sanitary product if necessary. The figure depicts the side-view of a fully assembled Protective Shield Blocker 100.

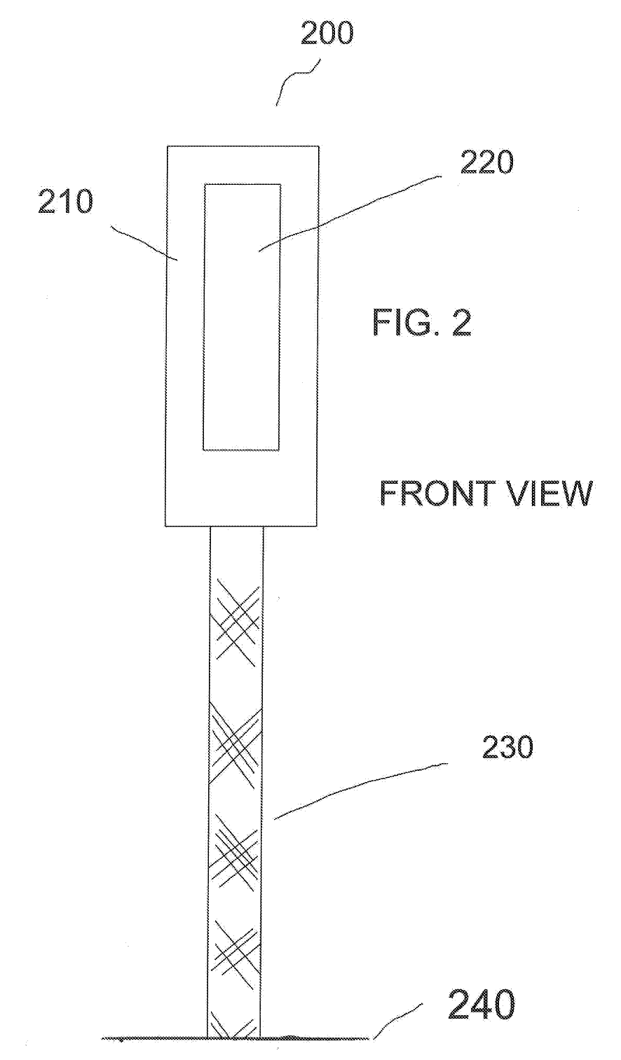

Turning now to FIG. 2, this figure presents a front-view of the pad section of the protective shield blocker for an exemplary view of the absorbent pad material 220 and the surrounding pad surface 210. The absorbent pad material 220 serves as the focal point for menstrual containment if the menstrual flow has breached an existing sanitary product such as a sanitary napkin. The remaining pad surface 210 acts as additional absorbent material to ensure that menstrual flow is adequately contained. The flexible cord 230 serves to connect the landing surface to the pad surface. The landing 240 serves as the primary attachment surface between an existing sanitary product and the Protective Shield Blocker.

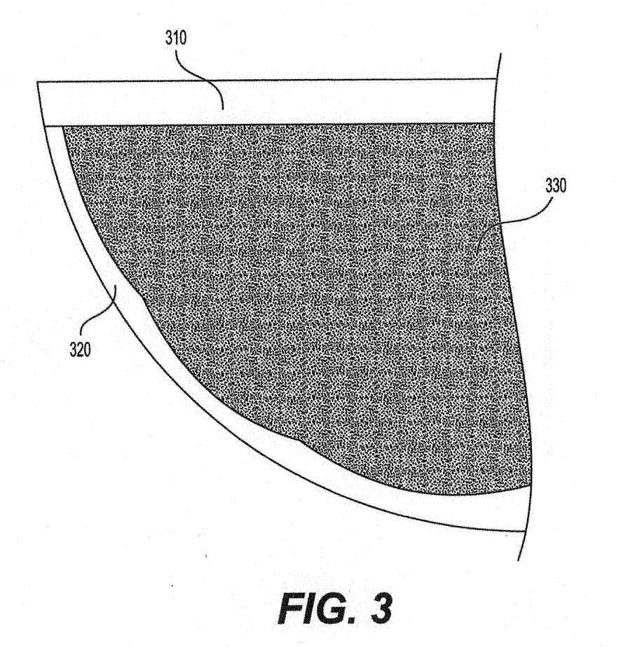

Turning now to FIG. 3, this figure presents a cross-sectional view of the Protective Shield Blocker 300 for an exemplary illustration of the upper permeable section 310 and the lower impermeable section 320. The schematic further presents an absorbent core material 330. The upper permeable section 310 allows fluids to pass through the upper material where it is absorbed by the core material 330. As the bodily fluids are absorbed the fluids are eventually arrested against the lower impermeable section 320 which acts as a barrier and does not allow fluids to pass beyond the outer surface of the Protective Shield Blocker 300.

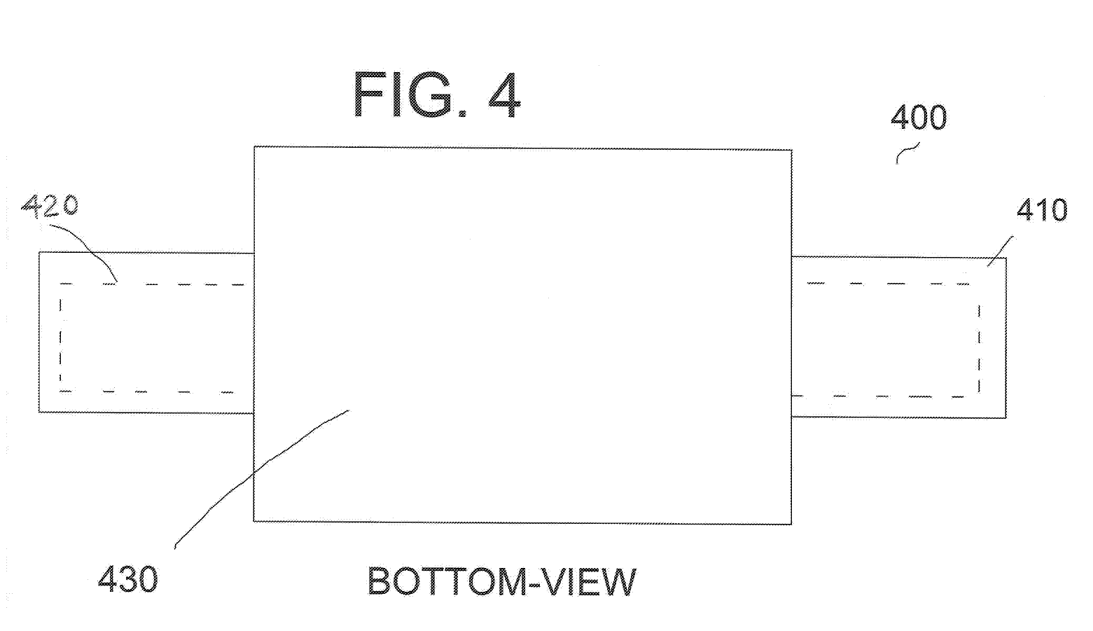

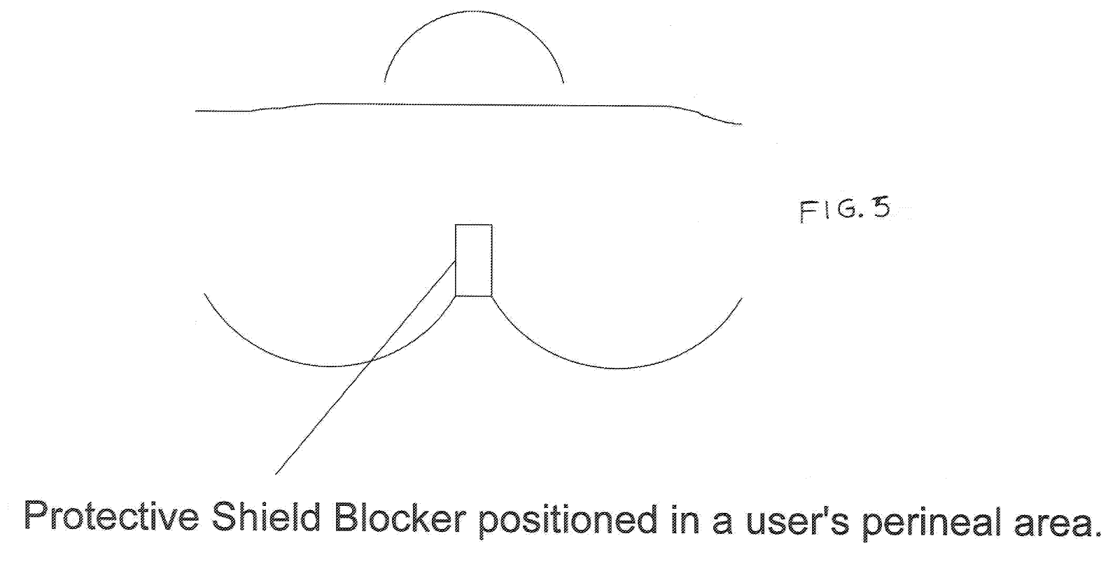

Turning now to FIG. 4, this figure presents a bottom view of the Protective Shield Blocker 400 for an exemplary illustration of the Protective Shield Blocker depicting the under body of the absorbent pad and landing with (transparent lines indicating the absorbent core material) 420. The schematic further presents a bottom view of the connecting surface of the landing 430. The landing 430 may be placed on the mating surface of another brand of sanitary products. The landing 430 forms a bond with the existing sanitary product.

Turning now to FIG. 5, this figure presents a view of a user's perineal area while the Protective Shield Blocker is secured between the wearer's buttocks. The schematic depicts what an embodiment of the Protective Shield Blocker would look like if positioned without the presence of the flexible cord. This depiction is merely intended to provide a better understanding of where the Protective Shield Blocker will be placed when in use.

I claim:

1. A female hygiene product for preventing menstrual back flow, comprising:
(a) a pad, wherein the pad forms a soft upper permeable liquid-absorbing panel having an external surface facing towards a user's body and an impermeable liquid-arresting lower panel opposing the upper panel having an external surface facing away from the user's body when the product is in use;
(b) an absorbent core material, wherein said absorbent core material is placed in a space between the soft upper panel and lower panel of said pad;
(c) a cord, wherein said cord connects to the lower panel of said pad;
(d) a landing attachment, wherein said landing attachment is connected to opposite end of said cord.

2. The female hygiene product according to claim 1; wherein the liquid-absorbing panel and said liquid-arresting panel each have an average caliper, the average caliper of said liquid-arresting panel being less than the average caliper of said liquid-absorbing panel.

3. The female hygiene product according to claim 2; wherein the average caliper of said liquid-absorbing panel and said liquid-arresting panel have a ratio ranging from about 3:1 to about 6:1.

4. The female hygiene product according to claim 1; wherein the pad surface further comprises a permeable liquid-absorbing upper panel that allows liquid to pass through a fiber membrane.

5. The female hygiene product according to claim 4; wherein said fiber membrane further comprises a transfer layer and the absorbent upper panel is continuous with the transfer layer.

6. The female hygiene product according to claim 1; wherein the pad surface further comprises an impermeable liquid-arresting lower panel that encapsulates any residual exiting liquid from the absorbing upper panel.

7. The female hygiene product according to claim 6, wherein the impermeable lower panel further comprises a transfer layer and the material of said liquid arresting section is continuous with the transfer layer.

8. The female hygiene product according to claim 1; wherein the absorbent core further occupies the space between the upper permeable panel and the impermeable lower panel.

9. The female hygiene product according to claim 8; wherein the absorbent core is made of a material that will allow liquid to pass through but allows the liquid to be transmitted through the material in a way to hold the liquid.

10. The female hygiene product according to claim 1; wherein the cord further comprises of a flexible material.

11. The female hygiene product according to claim 10; wherein said flexible cord has two opposing ends that attaches to the pad and the landing attachment.

12. The female hygiene product according to claim 1; wherein the landing attachment further comprises a panel surface that mates to the end of the cord that opposes the pad.

13. The female hygiene product according to claim 12; wherein the panel surface further comprises any suitable means of attaching the landing to an existing sanitary product.

14. The female hygiene product according to claim 1; wherein the pad thickness is between 0.5 cm and 25 cm.
